(19) United States
(12) Patent Application Publication
(10) Pub. No.: US 2026/0166303 A1
(43) Pub. Date: Jun. 18, 2026

(54) ELECTRODE DEVICE BASED ON MULTI-PHASE LIQUID BRIDGE AND USE METHOD THEREFOR

(71) Applicant: NANTONG UNIVERSITY, NANTONG (CN)

(72) Inventor: XIONGHENG BIAN, SUZHOU (CN)

(21) Appl. No.: 18/985,419

(22) Filed: Dec. 18, 2024

(51) Int. Cl. A61N 1/04

(52) U.S. Cl. CPC A61N 1/0496; A61N 1/0404

(57) ABSTRACT

A multi-phase fluid liquid bridge electrode device and a use method therefor are provided. The multi-phase fluid liquid bridge electrode device includes a flexible upper plate, a lower plate, and liquid bridges. The flexible upper plate is attached to and sealed with the lower plate. One side surface of the lower plate close to the flexible upper plate defines accommodating mechanisms configured to accommodate the liquid bridges. A connecting piece is disposed on one side surface of the flexible upper plate close to the lower plate. The liquid bridges stored in the accommodating mechanisms are respectively in contact with the connecting piece and the one side surface of the flexible upper plate close to the lower plate, so the lower plate, the liquid bridges, and the flexible upper plate are connected as a whole.

### TECHNICAL FIELD

The present disclosure relates to a field of bioelectric stimulation technology, and in particular to a multi-phase fluid liquid bridge electrode device and a use method therefor.

### BACKGROUND

History of treating diseases with drugs is long. Drug treatment mainly maintains health of a living body by supplementing certain chemicals that cannot be produced by the living body itself to the living body or by absorbing certain chemicals to maintain a physiological balance of the living body. An absorption rate of the drugs by the living body is an important treatment indicator. When a drug solution is used alone, permeability and absorption efficiency of the drug solution may be low, which causes drug waste. In addition, a distribution of the drugs in the living body may not be uniform enough and cannot be effectively concentrated in a target area, resulting in poor treatment effect, which increases a treatment time and increases a discomfort time.

Electrical stimulation is widely used in treatment of patients as a common rehabilitation treatment method. However, the electrical stimulation may cause immune reactions near biological tissues, especially allergic reactions and skin burns. The allergic reactions are generally caused by abnormal immune responses of individuals to biological electrical stimulation, which may cause symptoms such as urticaria and itching, and in severe cases, breathing difficulties or shock. Regarding to the skin burns, heat generated when an electric current passes through the living body, which may cause the skin burns, causing discomfort symptoms such as local redness, swelling, blisters, etc., and in severe cases, permanent skin damage may occur.

### SUMMARY

An object of the present disclosure is to provide an electrode device based on multi-phase liquid bridge and a use method therefor to solve defects in the prior art.

To achieve the above object, the present disclosure provides the electrode device based on multi-phase liquid bridge. This electrode device comprises a flexible upper plate, a lower plate, and liquid bridges.

The flexible upper plate is attached to and sealed with the lower plate. One side surface of the lower plate close to the flexible upper plate defines accommodating mechanisms configured to accommodate the liquid bridges. A connecting piece is disposed on one side surface of the flexible upper plate close to the lower plate. The liquid bridges stored in the accommodating mechanisms are respectively in contact with the connecting piece and the one side surface of the flexible upper plate close to the lower plate, so the lower plate, the liquid bridges, and the flexible upper plate are connected as a whole.

Furthermore, the lower plate comprises a substrate and a hydrogel layer. First holes are defined in the substrate. The first holes penetrate through the substrate from a top portion of the substrate to a bottom portion of the substrate. The hydrogel layer is fixedly disposed on the bottom portion of the substrate. Columns protruding from a surface of the hydrogel layer are disposed on a top portion of the hydrogel layer. The columns one-to-one pass through the first holes. Second holes are respectively defined in the columns, each of the second holes penetrates through a top portion of a corresponding one of the columns, and the accommodating mechanisms comprise the first holes and the columns.

Furthermore, the flexible upper plate comprises an insulating coating layer and a flexible film. The insulating coating layer is detachably mounted on a top portion of the substrate. The flexible film is fixedly mounted on a top portion of the insulating coating layer.

The connecting piece comprises electrodes fixedly mounted on a bottom portion of the flexible film. The electrodes pass through the insulating coating layer and extend to an outside of the insulating coating layer. Each of the electrodes is independently powered on/off.

Furthermore, the liquid bridges are conductive drug solutions.

Furthermore, a length of the substrate is 29 mm, a height of the substrate is 2 mm, and a width of the substrate is 19 mm. A diameter of each of the first holes in the top portion of the substrate is 1 mm.

Furthermore, a length of the hydrogel layer is 29 mm, a height of the hydrogel layer is 0.5 mm, and a width of the hydrogel layer is 19 mm. A height of each of the columns is 1.5 mm. A diameter of a top surface of each of the columns is 1 mm. A diameter of each of the second holes in the top portion of the corresponding one of the columns is 0.4 mm. A height of each of the columns is 1.5 mm.

Furthermore, a height of each of the electrodes is 0.2 mm, and a diameter of a top surface of each of the electrodes is 0.8 mm.

The present disclosure further provides a method for manufacturing the electrode device mentioned above. The method comprises steps A1-A7.

The step A1 comprises manufacturing a substrate by using a flexible resin through a three-dimensional (3D) printing technology.

The step A2 comprises manufacturing an auxiliary fixing structure by using the flexible resin through the 3D printing technology, and after the auxiliary fixing structure is obtained, inserting the auxiliary fixing structure into first holes of the substrate and configuring the auxiliary fixing structure as a base.

The step A3 comprises injecting an insulating hydrogel solution onto a surface of the substrate, and after the first holes are filled with the insulating hydrogel solution and the insulating hydrogel solution covers the surface of the substrate, performing ultraviolet exposure on the insulating hydrogel solution to enable the hydrogel solution being cured into a hydrogel layer.

The step A4 comprises removing the auxiliary fixing structure, and combing the substrate and the hydrogel layer to form the lower plate.

The step A5 comprises printing electrodes on a bottom portion of a flexible film by a print circuit board (PCB) printing flexible electrode technology, and covering an insulating coating layer on a bottom surface of the flexible film except for positions where the electrodes are located to obtain the flexible upper plate.

The step A6 comprises preparing the liquid bridges.

The step A7 comprises dripping the liquid bridges into the first holes in the lower plate, and covering the flexible upper plate on the lower plate to obtain the electrode device.

The present disclosure further provides a use method for the electrode device mentioned above. The use method comprises steps S1-S4.

The step S1 comprises attaching the lower plate to an attachment area, where electrical stimulation and drug treatment are required for a patient.

The step S2 comprises dropping the liquid bridges on the lower plate to enable the liquid bridges to enter the accommodating mechanisms.

The step S3 comprises covering the flexible upper plate on the lower plate, and detachably connecting the flexible upper plate to the substrate through an insulating coating layer, enabling the lower plate, the liquid bridges and the flexible upper plate being connected as a whole.

The step S4 comprises applying an electrical stimulation signal to the flexible upper plate, so that the electrical signal forms a loop and the electrical signal provides electrical stimulation therapy to the patient through an assistance of the liquid bridges.

Compared with the prior art, in the electrode device and the use method of the present disclosure, the conductive drug solutions are added to assist drug therapy while achieving electrical stimulation therapy, which complements each other. In addition, the flexible upper plate and the lower plate have better adhesion to the attachment area. The electrodes conduct charge through the liquid bridges to achieve the electrical stimulation therapy, which avoid immune reactions near the attachment area (i.e., biological tissues), improve permeability and absorption efficiency of drugs, make a distribution of the drugs in a living body uniform, improve a treatment effect, and shorten a treatment time. For attachment area within a coverage of the electrode device, the electrodes are able to selectively stimulate and treat the attachment area with drugs.

### BRIEF DESCRIPTION OF DRAWINGS

In order to clearly describe technical solutions in the embodiments of the present disclosure, the following will briefly introduce the drawings that need to be used in the description of the embodiments or the prior art. Apparently, the drawings in the following description are merely some of the embodiments of the present disclosure, and those skilled in the art are able to obtain other drawings according to the drawings without contributing any inventive labor.

FIG. 1 is a schematic diagram showing a manufacturing process of an electrode device according to one embodiment of the present disclosure.

FIG. 2 is a schematic diagram showing a cross-segmental structure of the electrode device according to one embodiment of the present disclosure.

FIG. 3 is a schematic diagram showing a size of a substrate according to one embodiment of the present disclosure.

FIG. 4 is a schematic diagram showing a size of an auxiliary fixing structure according to one embodiment of the present disclosure.

FIG. 5 is a schematic diagram showing a size of a hydrogel layer according to one embodiment of the present disclosure.

FIG. 6 is a schematic diagram showing a manufacturing process of a flexible upper plate according to one embodiment of the present disclosure.

FIG. 7 is a schematic diagram showing a size of the flexible upper plate according to one embodiment of the present disclosure.

FIG. 8 is a schematic diagram showing the electrode device of the present disclosure in a configuration of use and conventional electrical stimulation electrodes in a configuration of use.

FIG. 9 is a flow chart showing a use process of the electrode device according to one embodiment of the present disclosure.

FIG. 10 is a schematic diagram showing a use method of the electrode device according to one embodiment of the present disclosure.

FIG. 11 is a schematic diagram showing a comparison of a gripping strength of hind limbs of a rat before and after using the electrode device according to one embodiment of the present disclosure.

FIG. 12 is a schematic diagram of symmetrical point coordinates of the rat according to one embodiment of the present disclosure.

FIG. 13 is a schematic diagram of spinal cord segments and spinal segments according to one embodiment of the present disclosure.

FIG. 14 is a schematic diagram of the spine exposed in a rat spinal cord injury treatment experiment according to one embodiment of the present disclosure.

FIG. 15 is a schematic diagram of the spinal cord exposed in the rat spinal cord injury treatment experiment according to one embodiment of the present disclosure.

FIG. 16 is a schematic diagram of an insulation area of the electrode device according to one embodiment of the present disclosure.

Reference numbers in the drawings:
1—substrate; 2—auxiliary fixing structure; 3—hydrogel layer; 4—flexible upper plate; 5—flexible film; 6—electrode; 7—insulating coating layer; 8—attachment area; 9—liquid bridge.

### DETAILED DESCRIPTION

In order to enable those skilled in the art to better understand the technical solutions of the present disclosure, the present disclosure is further described in detail below with reference to the accompanying drawings.

### Embodiment 1

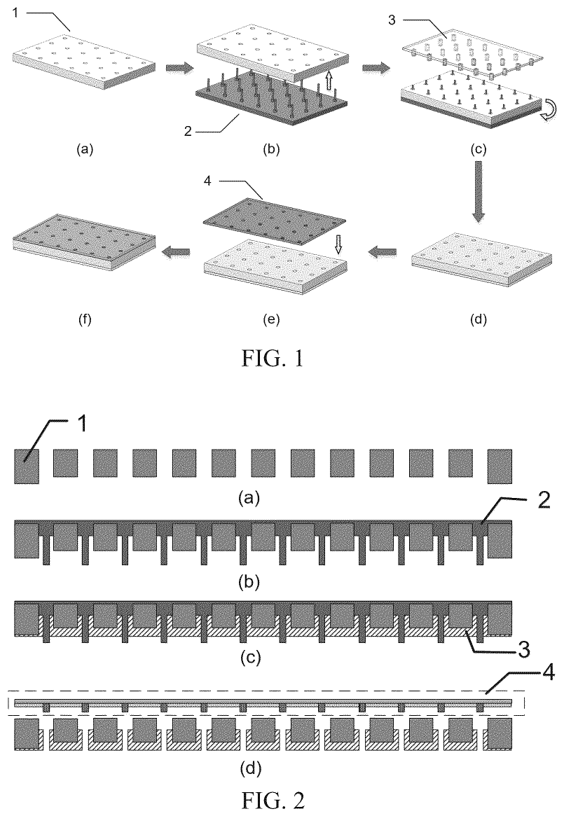

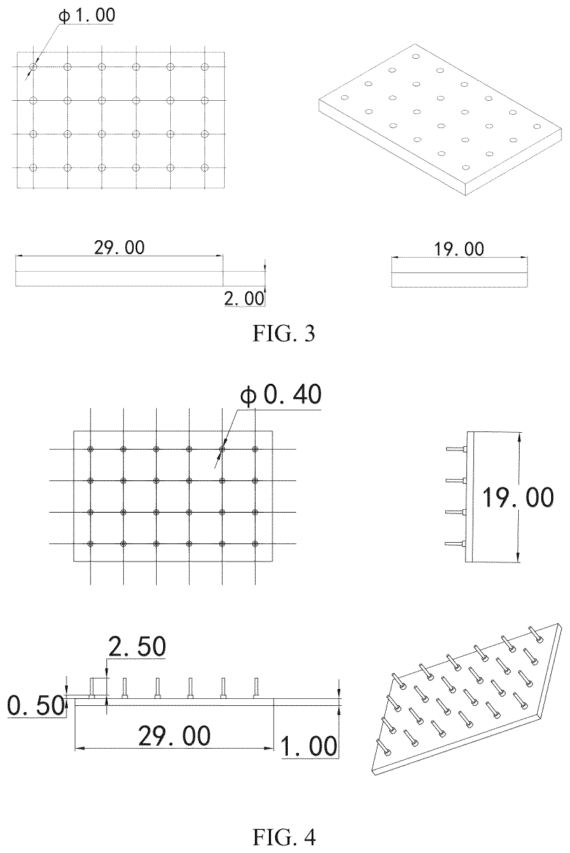

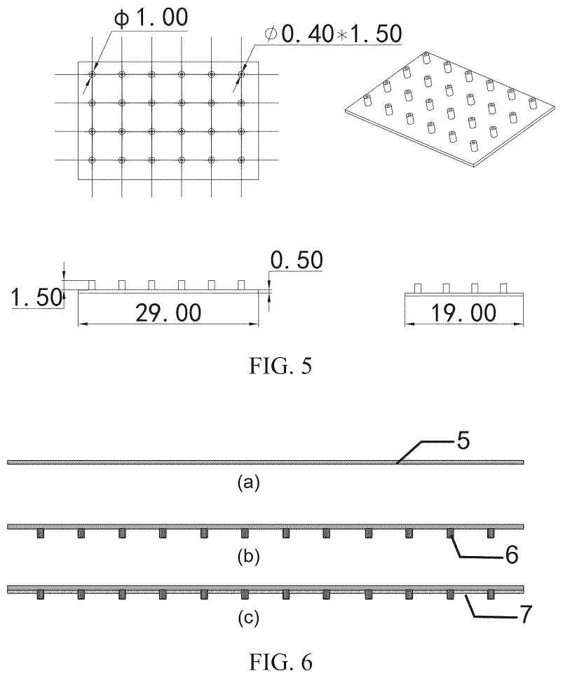

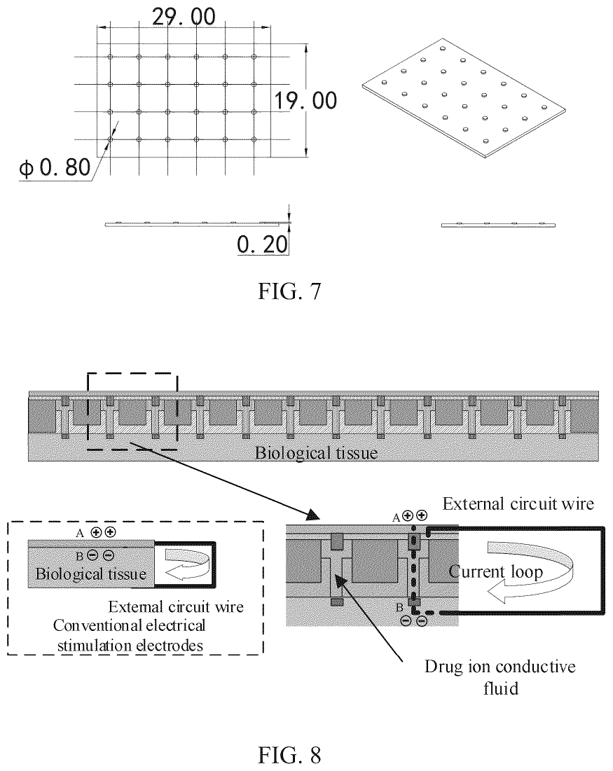

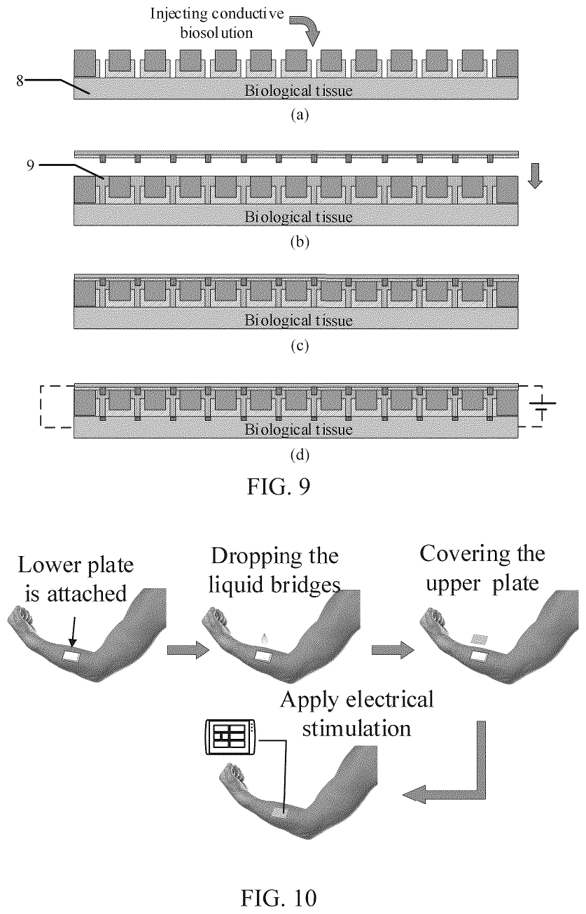

As shown in FIGS. 1-9 and 16, the present disclosure provides an electrode device. The multi-phase fluid liquid bridge electrode device comprises a flexible upper plate 4, a lower plate, and liquid bridges 9.

The flexible upper plate 4 is attached to and sealed with the lower plate. One side surface of the lower plate close to the flexible upper plate 4 defines accommodating mechanisms configured to accommodate the liquid bridges 9. A connecting piece is disposed on one side surface of the flexible upper plate 4 close to the lower plate. The liquid bridges 9 stored in the accommodating mechanisms are respectively in contact with the connecting piece and the one side surface of the flexible upper plate 4 close to the lower plate, so the lower plate, the liquid bridges 9, and the flexible upper plate 4 are connected as a whole.

Specifically, the flexible upper plate 4 is detachably connected to the lower plate and is sealed with the lower plate when being attached to the lower plate, so that when a circuit of the flexible upper electrode plate 4 fails, the flexible upper electrode plate 4 is allowed to be replaced, thereby preventing treatment interruption and avoiding affecting a treatment effect.

The lower plate comprises a substrate 1 and a hydrogel layer 3, and first holes are defined in the substrate 1. The first holes penetrate through the substrate 1 from a top portion of the substrate 1 to a bottom portion of the substrate 1. The hydrogel layer 3 is fixedly disposed on the bottom portion of the substrate 1. Columns protruding from a surface of the hydrogel layer 3 are disposed on a top portion of the hydrogel layer 3. The columns one-to-one pass through the first holes. Second holes are respectively defined in the columns, each of the second holes penetrates through a top portion of a corresponding one of the columns, and the accommodating mechanisms comprise the first holes and the columns.

A length of the substrate 1 is 29 mm, a height of the substrate 1 is 2 mm, and a width of the substrate 1 is 19 mm. A diameter of each of the first holes in the top portion of the substrate 1 is 1 mm. A length of the hydrogel layer 3 is 29 mm, a height of the hydrogel layer 3 is 0.5 mm, and a width of the hydrogel layer 3 is 19 mm. A height of each of the columns is 1.5 mm. A diameter of a top surface of each of the columns is 1 mm. A diameter of each of the second holes in the top portion of the corresponding one of the columns is 0.4 mm. A height of each of the columns is 1.5 mm.

Specifically, the substrate 1 is made from a flexible resin, and the substrate 1 is made by 3D printing. The flexible resin has a high degree of flexibility, and is allowed to be bent, folded, or even twisted without being easily broken or damaged. The flexible resin has a soft touch, which brings a comfortable wearing experience to a patient, thereby having better adhesion to a living body. The flexible resin has a low density, so the flexible resin is very light and is easy to carry. Moreover, the flexible resin has good chemical stability and is able to resist corrosion from a variety of chemical substances, so the flexible resin is allowed to be used in a variety of chemical environments. The substrate 1 is made from the flexible resin, which is convenient for accommodating the liquid bridges 9, is easy to process, and is able to meet shape and size requirements of the multi-phase fluid liquid bridge electrode device.

Hydrogels have good biocompatibility and biodegradability. The hydrogels are closer to living tissues than any synthetic biomaterials and are similar to extracellular matrix in nature, which makes the hydrogels widely used in a medical field, such as drug delivery, tissue engineering, wound dressing preparations, etc.

The flexible upper plate 4 comprises an insulating coating layer 7 and a flexible film 5. The insulating coating layer 7 is detachably mounted on a top portion of the substrate 1. The flexible film 5 is fixedly mounted on a top portion of the insulating coating layer 7.

The connecting piece comprises electrodes 6 fixedly mounted on a bottom portion of the flexible film 5. The electrodes 6 pass through the insulating coating layer 7 and extend to an outside of the insulating coating layer 7. Each of the electrodes 6 is independently powered on/off. A height of each of the electrodes 6 is 0.2 mm, and a diameter of a top surface of each of the electrodes 6 is 0.8 mm.

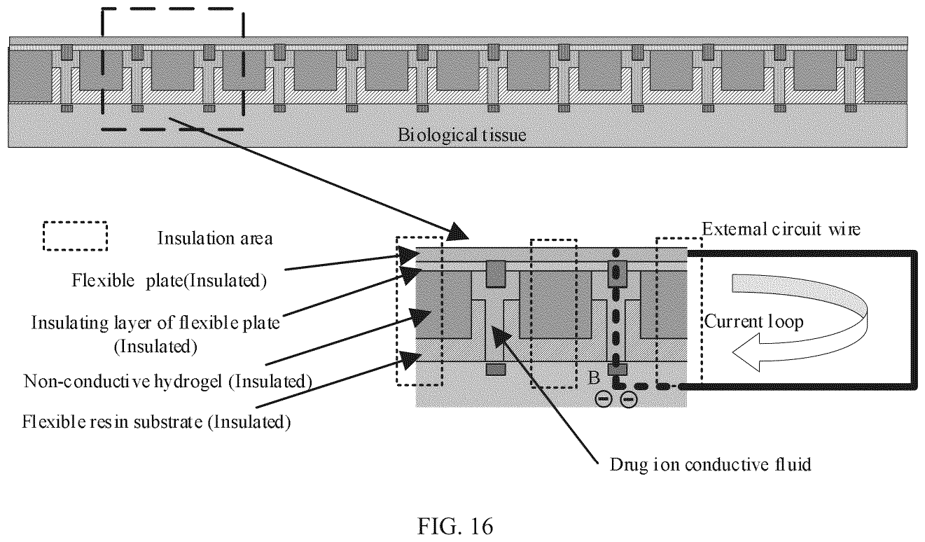

Specifically, the flexible film 5 is made from polyimide. The polyimide has good chemical stability, moisture, and heat resistance, is insoluble in organic solvents, is corrosion-resistant and hydrolysis-resistant, and has high insulation performance. The flexible film 5 is used together with the insulating coating layer 7 and the electrodes 6 to divide areas corresponding to the electrodes 6 into independent circuits. Different currents are respectively applied to the independent circuits to effectively avoid mutual interference between the electrodes 6 and reduce a possibility of safety accidents such as leakage of the liquid bridges, short circuit, and thermal runaway. Insulating areas are shown in FIG. 16.

The liquid bridges 9 are conductive drug solutions.

Specifically, for treatment of spinal cord injury, the conductive drug solutions are prepared by adding 1 mL of a 200 ng/mL methyl cobalamin solution to 1 mL of normal saline.

### Embodiment 2

As shown in FIGS. 1-9, the present disclosure further provides a method for manufacturing the multi-phase fluid liquid bridge electrode device mentioned above. The method comprises steps A1-A7.

The step A1 comprises manufacturing a substrate 1 by using a flexible resin through a three-dimensional (3D) printing technology.

The step A2 comprises manufacturing an auxiliary fixing structure 2 by using the flexible resin through the 3D printing technology, and after the auxiliary fixing structure 2 is obtained, inserting the auxiliary fixing structure 2 into first holes of the substrate 1 and configuring the auxiliary fixing structure 2 as a base.

The step A3 comprises injecting an insulating hydrogel solution onto a surface of the substrate 1, and after the first holes are filled with the insulating hydrogel solution and the insulating hydrogel solution covers the surface of the substrate 1, performing ultraviolet exposure on the insulating hydrogel solution to enable the hydrogel solution being cured into a hydrogel layer 3.

The step A4 comprises removing the auxiliary fixing structure 2, and combing the substrate 1 and the hydrogel layer 3 to form the lower plate.

The step A5 comprises printing electrodes 6 on a bottom portion of a flexible film 5 by a print circuit board (PCB) printing flexible electrode technology, and covering an insulating coating layer 7 on a bottom surface of the flexible film 5 except for positions where the electrodes 6 are located to obtain the flexible upper plate 4.

The step A6 comprises preparing the liquid bridges 9.

The step A7 comprises dripping the liquid bridges 9 into the first holes in the lower plate, and covering the flexible upper plate 4 on the lower plate to obtain the electrode device.

### Embodiment 3

As shown in FIG. 10, based on Embodiment 1, the present disclosure further provides a use method for the electrode device mentioned above. The use method comprises steps S1-S4.

The step S1 comprises attaching the lower plate to an attachment area 8, where electrical stimulation and drug treatment are required for a patient.

The step S2 comprises dropping the liquid bridges 9 on the lower plate to enable the liquid bridges 9 to enter the accommodating mechanisms.

The step S3 comprises covering the flexible upper plate 4 on the lower plate, and detachably connecting the flexible upper plate 4 to the substrate 1 through an insulating coating layer 7, enabling the lower plate, the liquid bridges 9 and the flexible upper plate 4 being connected as a whole.

The step S4 comprises applying an electrical stimulation signal to the flexible upper plate 4, so that the electrical signal forms a loop and the electrical signal provides electrical stimulation therapy to the patient through an assistance of the liquid bridges 9.

### Embodiment 4

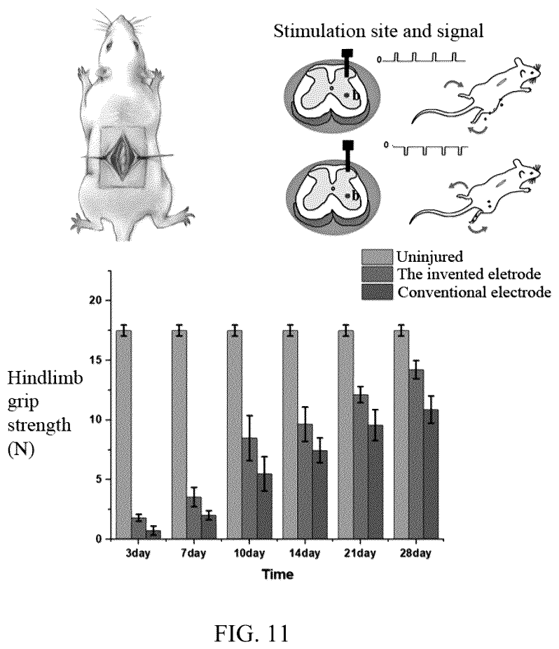

As shown in FIGS. 11-15, the embodiment provides a specific experimental demonstration of a therapeutic effect of the electrode device on a basis of the embodiment 1. A spinal cord injury rat treatment experiment is as follow.

Surgical preparation is as follow. First, surgical instruments are sterilized with high temperature and a surgical blade thereof is replaced. Then, a thermostat of an operating table is turned on and maintained at 37° C. to maintain a body temperature of a rat. The rat is anesthetized by intraperitoneal injection of anesthetic (0.2 mL/100 g). The rats is observed whether the rat is fully anesthetized. After the rat is fully anesthetized, hair on a back of the rat is shaved with an animal electric shaver.

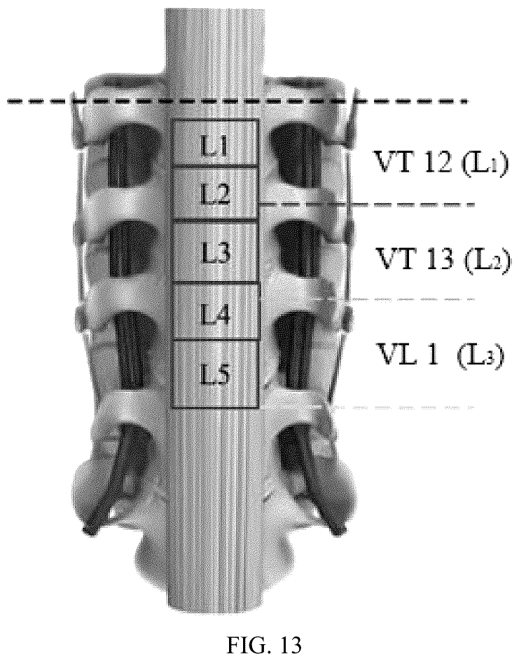

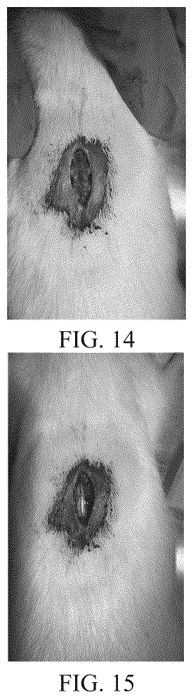

A surgical procedure is shown in FIG. 13. First, T9-T13 segments of the spine of the rat is found, and a midline incision is made on a back skin with a scalpel to expose spinal segments VT9 and VT12-VL1 of the rat, and spinal cord segments L1-L5 are determined. As shown in FIGS. 14-15, muscles on a surface of a vertebral plate of the rat are removed with hemostatic forceps, and a spinal cord of the rat is injured at a selected spinal segment (VT9) by the scalpel, causing paralysis of lower limbs the rat.

A site (CPG site) inducing contraction and extension of the lower limbs is found by a stimulation electrode, the lower plate that is flexible is attached to a nearby muscle of the CPG site, then a treatment solution is dripped on the electrodes, the flexible upper plate is covered and then electrical stimulation is applied. During the electrical stimulation, constant pulses of positive or negative polarity (with a pulse width of 200 μs, a frequency of 33 Hz, a pulse interval 30 ms; of the same polarity, a positive and negative pulse train interval of 1.5 s; a stimulation duration of 3 s; and an intensity of 300-300 μA) are provided by an isolated stimulator.

The operation lasts about 2-3 hours. During the operation, the body temperature and respiratory rate of the rat are monitored simultaneously, and an anesthesia status of the rat is observed. If necessary, a small amount of anesthetic is added during the operation.

Postoperative process: The rat with spinal cord injury is implanted with the electrode device to observe recovery. A pair of potentialfieldapproach-coated (PFA-coated) tungsten wire electrodes are sutured on a selected ventral side for bipolar recording. The electrode device is connected to the CPG site through biocompatible gutta-percha. The, the rat skin is sutured and thoroughly cleaned with saline.

After suturing, the rat is placed in a heated incubator until it regained consciousness. The rat with spinal cord injury is then trained with intraspinal microstimulation for four weeks, with 20 minutes of training per day, 5 days a week. After one month, a motion function of the rat is observed for determining recovery of the rat.

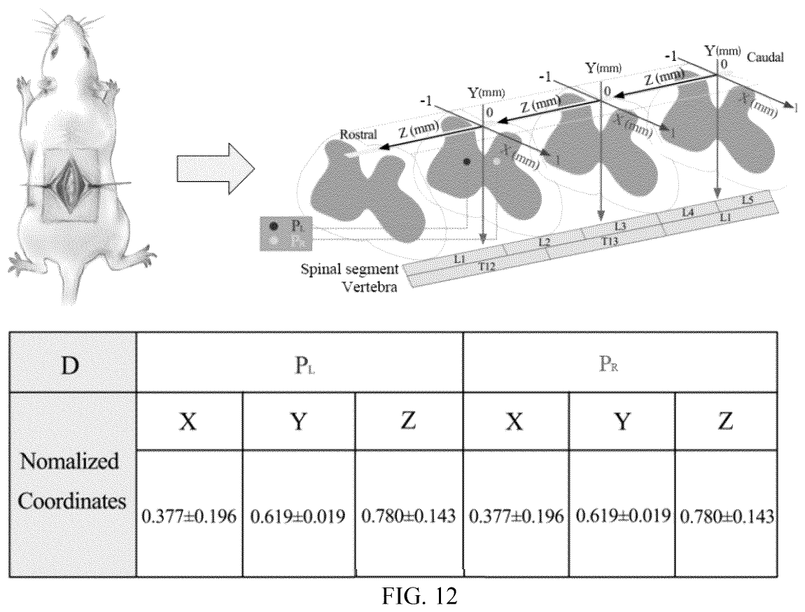

For the rat with spinal cord injury, a hind limb grip strength is served as a measure of rat health, and two symmetrical sites are selected for treatment. Coordinates of the two symmetrical sites are shown in FIG. 12, and a treatment result thereof is compared with a treatment results obtained using conventional electrical stimulation electrodes. As shown in FIG. 11, experimental results show that the two symmetrical sites have similar treatment effects, and compared with the conventional electrical stimulation electrodes, the electrodes of the multi-phase fluid liquid bridge electrode device has a better treatment effect.

While certain exemplary embodiments of the present disclosure are described above, it is apparent to those skilled in the art that modifications may be made to the described embodiments in a variety of different ways without departing from the spirit and scope of the present disclosure. Therefore, the above drawings and description are illustrative in nature and should not be construed as limiting the scope of the claims of the present disclosure.

What is claimed is:

1. An electrode device, comprising:
a flexible upper plate;
a lower plate; and
liquid bridges;
wherein the flexible upper plate is attached to and sealed with the lower plate, one side surface of the lower plate close to the flexible upper plate defines accommodating mechanisms configured to accommodate the liquid bridges, a connecting piece is disposed on one side surface of the flexible upper plate close to the lower plate, and the liquid bridges stored in the accommodating mechanisms are respectively in contact with the connecting piece and the one side surface of the flexible upper plate close to the lower plate, so the lower plate, the liquid bridges, and the flexible upper plate are connected as a whole.

2. The electrode device according to claim 1, wherein the lower plate comprises a substrate and a hydrogel layer, first holes are defined in the substrate, the first holes penetrate through the substrate from a top portion of the substrate to a bottom portion of the substrate, the hydrogel layer is fixedly disposed on the bottom portion of the substrate, columns protruding from a surface of the hydrogel layer are disposed on a top portion of the hydrogel layer, the columns one-to-one pass through the first holes, second holes are respectively defined in the columns, each of the second holes penetrates through a top portion of a corresponding one of the columns, and the accommodating mechanisms comprise the first holes and the columns.

3. The electrode device according to claim 1, wherein the flexible upper plate comprises an insulating coating layer and a flexible film, the insulating coating layer is detachably mounted on a top portion of the substrate, and the flexible film is fixedly mounted on a top portion of the insulating coating layer;
wherein the connecting piece comprises electrodes fixedly mounted on a bottom portion of the flexible film, the electrodes pass through the insulating coating layer and extend to an outside of the insulating coating layer, and each of the electrodes is independently powered on/off.

4. The electrode device according to claim 1, wherein the liquid bridges are conductive drug solutions.

5. The electrode device according to claim 2, wherein a length of the substrate is 29 mm, a height of the substrate is 2 mm, a width of the substrate is 19 mm, and a diameter of each of the first holes in the top portion of the substrate is 1 mm.

6. The electrode device according to claim 2, wherein a length of the hydrogel layer is 29 mm, a height of the hydrogel layer is 0.5 mm, a width of the hydrogel layer is 19 mm, a height of each of the columns is 1.5 mm, a diameter of a top surface of each of the columns is 1 mm, a diameter of each of the second holes in the top portion of the corresponding one of the columns is 0.4 mm, and a height of each of the columns is 1.5 mm.

7. The electrode device according to claim 3, wherein a height of each of the electrodes is 0.2 mm, and a diameter of a top surface of each of the electrodes is 0.8 mm.

8. A method for manufacturing a multi-phase fluid liquid bridge electrode device, comprising:
a step A1: manufacturing a substrate by using a flexible resin through a three-dimensional (3D) printing technology;
a step A2: manufacturing an auxiliary fixing structure by using the flexible resin through the 3D printing technology, and after the auxiliary fixing structure is obtained, inserting the auxiliary fixing structure into first holes of the substrate and configuring the auxiliary fixing structure as a base;
a step A3: injecting an insulating hydrogel solution onto a surface of the substrate, and after the first holes are filled with the insulating hydrogel solution and the insulating hydrogel solution covers the surface of the substrate, performing ultraviolet exposure on the insulating hydrogel solution to enable the hydrogel solution being cured into a hydrogel layer;
a step A4: removing the auxiliary fixing structure, and combing the substrate and the hydrogel layer to form a lower plate;
a step A5: printing electrodes on a bottom portion of a flexible film by a print circuit board (PCB) printing flexible electrode technology, and covering an insulating coating layer on a bottom surface of the flexible film except for positions where the electrodes are located to obtain a flexible upper plate;
a step A6: preparing liquid bridges; and
a step A7: dripping the liquid bridges into the first holes in the lower plate, and covering the flexible upper plate on the lower plate to obtain the electrode device.

9. A use method for an electrode device comprising:
a step S1: attaching a lower plate to an attachment area where electrical stimulation and drug treatment are required for a patient;
a step S2: dropping liquid bridges on the lower plate to enable the liquid bridges to enter accommodating mechanisms;
a step S3: covering a flexible upper plate on the lower plate, and detachably connecting the flexible upper plate to a substrate through an insulating coating layer, enabling the lower plate, the liquid bridges, and the flexible upper plate being connected as a whole;
a step S4: applying an electrical stimulation signal to the flexible upper plate, so that an electrical signal forms a loop and the electrical signal provides electrical stimulation therapy to the patient through an assistance of the liquid bridges.
